(19) United States
(12) Patent Application Publication
(10) Pub. No.: US 2009/0036811 A1
(43) Pub. Date: Feb. 5, 2009

(54) Orthosis casting form and method of making the same

(75) Inventors: Roy Lidtke, Elberon, IA (US); Gavin Hassell, Murrysville, PA (US)

Correspondence Address: THE WEBB LAW FIRM, P.C., 700 KOPPERS BUILDING, 436 SEVENTH AVENUE, PITTSBURGH, PA, 15219, US

(73) Assignee: Tower Orthopedic Designs, Inc., Pittsburgh, PA (US)

(21) Appl. No.: 11/888,564

(22) Filed: Aug. 1, 2007

(51) Int. Cl. A61F 5/00; A61F 13/00

(52) U.S. Cl. 602/6; 602/46

(57) ABSTRACT

A logic circuit that can reconfigure its functions in a nonvolatile manner and a single-electron transistor to be used in the logic circuits are provided. The logic circuit has a single-electron spin transistor that includes: a source; a drain; an island that is provided between the source and the drain, and has tunnel junctions between the island and the source and drain; and a gate that is capacitively coupled to the island. In this logic circuit, at least one of the source, the drain, and the island includes a ferromagnetic material having a variable magnetization direction.

### BACKGROUND OF THE INVENTION

1. Field of the Invention

The present invention relates to an orthosis casting form and, more particularly, relates to such a form comprised of dual density foam and a method of producing the same.

2. Description of Related Art

Pronation of the human foot involves an inward rolling motion of the foot as it contacts the ground during a gait cycle. Supination involves an outward rolling of the foot as it contacts the ground during a gait cycle. Pronation and supination of the foot normally occur during the gait cycle and are generally observed as rotation of the heel bone or calcaneus. In particular, during pronation, the heel rotates outwardly and during supination, the heel rotates inwardly.

Excessive pronation or supination of the foot is undesirable and may cause discomfort and injury. Common maladies resulting from excessive foot motion include heel pain, bunions, hammertoes, neromas, knee pain, shin splints and stress fractures.

Attempts have been made to provide devices for counteracting excessive pronation and/or supination and for treating symptoms associated with these conditions.

FIG. 1 shows the calcaneus 10 of a right foot 11 in a neutral position that is aligned properly with the adjacent talus 12 (FIG. 6) and corresponding leg bone 13. FIG. 2 illustrates the calcaneus 10 in a pronated position which many times is the result of a weak or fallen arch 14. FIG. 3 illustrates a calcaneus 10 in an under-pronated condition which is also known as supination. Directing attention to FIGS. 2 and 3, the area in which the arch is located is illustrated by reference number 14. These illustrations emphasize the importance of proper foot alignment to minimize potential pathologies resulting from abnormal biomechanical forces.

The most common way of addressing biomechanical problems, such as excessive pronation and supination, is through the use of foot orthosis often referred to as orthotics, which are custom designed forms which fit within a shoe to provide specialized support to the foot of the user. Each orthotic is designed specifically for the foot of an individual. Therefore, an exact impression of the topographical surface of the end user's foot is critical to the successful manufacture of custom foot orthotics. Those skilled in the art refer to a copy of a user's foot as a positive while the mating topographical surface is referred to as the negative. FIG. 4 illustrates a standard orthotic casting form 20 comprised of a crushable foam material 22 such as phenolic foam having a closed-cell structure. The form 20 has a top 21, a bottom 23 and sides 24, 25, 26, 27 therebetween. The foam 22 of the form 20 deforms when a weight is applied to it. In particular, with respect to the form 20, a user places his/her foot 11 on the top 21 of the form 20 as illustrated in FIG. 4. The user then applies weight to his/her foot 11 and crushes the foam 22 from the top 21 of the form 20. However, the foam 22 is not resilient and, therefore, maintains its crushed shape such that when the user removes his/her foot 11 from the form 20, an impression of the topographical surface and outline of the bottom of the foot 11 remains.

However, unfortunately, when using a standard crushable foam block, a user gets an impression of his/her foot 11 using the crushable foam 22 of the form 20, and the biomechanical deficiency of the foot manifests itself within the impression given by the foot 11. That is, in this partial to full weight-bearing state the foot falls into the same position that created the original condition. For that reason, an orthotic fabricated from the impression in the standard form 20 must be further modified by a fabricator to compensate for the biomechanical deficiency.

An apparatus and a method are needed, whereby the impression of a foot 11 using a crushable foam form 20 accurately represents an impression of the foot 11 in a corrected position. An orthotic, which will eventually be produced utilizing this impression, may then be fabricated without further manipulation of the impression in the form 20.

### SUMMARY OF THE INVENTION

In one embodiment of the subject invention, an orthosis casting form is comprised of a block of crushable foam, wherein the block has a top and a bottom, with sides therebetween. The top has a central region adapted to receive the foot of a user, such that under the weight of the user, the foam is crushed and provides an imprint of the topographical surfaces of the bottom and sides of the user's foot. The block is made up of at least two different densities of crushable foam.

A second embodiment of the subject invention is directed to a method of making an orthosis casting form, beginning with a block of single density crushable foam having a top and a bottom with sides therebetween. The top has a central region adapted to receive the foot of a user such that under the weight of the user, the foam is crushed and provides an imprint of the bottom of the user's foot. The method comprises the steps of removing a portion of the foam from the bottom in the region proximate to the forward portion of the lateral column and replacing at least part of the removed portion with a segment of crushable foam having a greater density, thereby providing greater resistance to penetration of the foam by the forward portion of the lateral column than to the other metatarsals.

### BRIEF DESCRIPTION OF THE DRAWINGS

FIGS. 1, 2 and 3 are sketches illustrating the rear view of a right foot with a calcaneus in a neutral position in FIG. 1, a pronated position in FIG. 2, and a supinated position in FIG. 3;

FIGS. 4 and 5 are sketches illustrating the sequence by which a user produces an impression using a crushable foam form;

FIG. 6 is a top view of a right foot illustrating the bones of the foot;

FIG. 7 is a side view of the right foot illustrating the bones of the foot;

FIG. 8 is a top perspective view of one embodiment of the subject invention;

FIG. 9 is an end view of the FIG. 8 embodiment;

FIG. 10 is a side perspective view of the FIG. 8 embodiment;

FIG. 11 is another side perspective view of the FIG. 8 embodiment;

FIG. 12 is a side view of the FIG. 8 embodiment;

FIG. 13 is a bottom view of the FIG. 8 embodiment;

FIG. 14 is a bottom perspective view of the FIG. 8 embodiment;

FIG. 15 is another end view but from the opposite end of that illustrated in FIG. 14;

FIG. 16A is an end perspective view illustrating a wedge at the rear end of the form in accordance with a second embodiment of the subject invention;

FIG. 16B is a perspective view identical to FIG. 16A but with the cavity filled with a setting foam;

FIG. 17 is another perspective view of the form illustrated in FIG. 16;

FIG. 18 is yet another perspective view of the form illustrated in FIG. 16, but including hidden lines to more fully illustrate the wedges; and

FIG. 19 is yet another perspective view of a form showing a plug instead of a wedge to provide stiffness.

### DESCRIPTION OF THE PREFERRED EMBODIMENTS

The Applicants have discovered that by using materials of different density for the fabrication of the form 20, the manner in which the foot orients itself within the form 20 may be controlled, such that the impression within the form more closely resembles the shape of the finally produced orthotic.

Throughout this application, reference is made to the parts of a foot with respect to the form 120. It should be appreciated that the form 120 is sized so that a typical foot, when placed in the central region of the form 120, will be properly located on the form 120 so that the parts of the foot will contact the appropriate locations on the form 120. However, the form 120 will be marked to define the front and back so that a user will not place his/her foot in the wrong direction, which would diminish the benefits of the subject invention.

Directing attention to FIGS. 6 and 7, the human foot has a number of bones including the calcaneus 10, talus 12, navicular 15, cuneiforms 16a, 16b, 16c, cuboid 18 and the first through fifth metatarsals 125, 126, 127, 128, 129, respectively. The medial column 30 for purposes of this discussion, includes the talus 12, navicular 15, the cuneiforms 16a, 16b, 16c, and the first, second and third metatarsals 125, 126, 127. The lateral column 40 for purposes of this discussion involves the calcaneus 10, cuboid 18 and the fourth and fifth metatarsals 128, 129. Each of the medial column 30 and the lateral column 40 move in a pivoting motion about the calcaneus 10 such that the forward portion 35 of the medial column and the forward portion 45 of the lateral column 40 (i.e., forward of the calcaneus 10) pivot up and down.

When the forward portion 45 of the lateral column 40 is pivoted upward, the mid-foot locks and the foot 11 becomes relatively rigid so that the individual may walk. The inside column of the foot 11 is referred to as the first ray and comprises the first metatarsal and big toe. The first ray has an independent axis of motion similar to the thumb in a human hand and, therefore, moves up when an application of force is applied from the bottom. The outside of the foot 11 is referred to as the fifth ray and its motion and function mirrors the medial column. In normal gait the outside of the heel strikes the ground first and bears weight quickly followed by the fifth ray and lateral column 40 of the foot 11. The load across the lateral column 40 forces the joints in this segment to produce an osseous restraining mechanism, often referred to as a “locked position”, so that the forces can be transmitted to adjoining structures. An ideal orthosis mimics the geometry of the foot 11 in this “locked position”. However, using a standard crushable foam form, the foam is significantly crushed before the foot 11 is in the “locked position” such that the negative imprinted in the form 120 does not represent the bio-mechanically correct position of the foot 11. During this time, the first ray is being pulled down and stabilized by muscles with the net effect of a stable foot structure that minimizes pronation of the foot. In a nonweight-bearing position, if the foot 11 were to dangle, the fifth metatarsal 129 would dangle at an elevation lower than the first metatarsal 125. As the foot 11 is placed in crushable foam, the fifth ray and lateral column stays lower than the first ray since the single density light foam cannot place sufficient force on the foot to load the lateral column. The resulting impression, or positive, captures the shape of the foot in an incorrect position. Similarly, to reduce pronation or rolling motion of the foot 11 when the user's foot is in the foam 122, greater force must be applied to the medial or inside of the heel area.

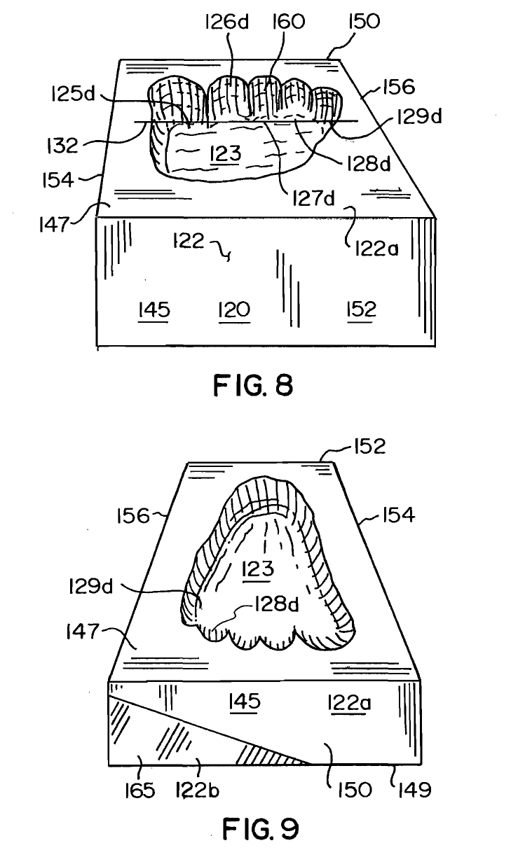

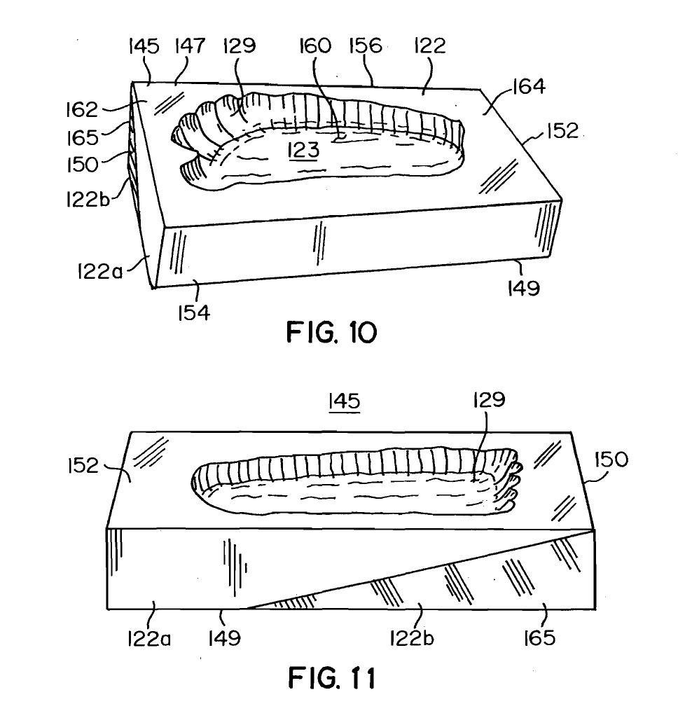

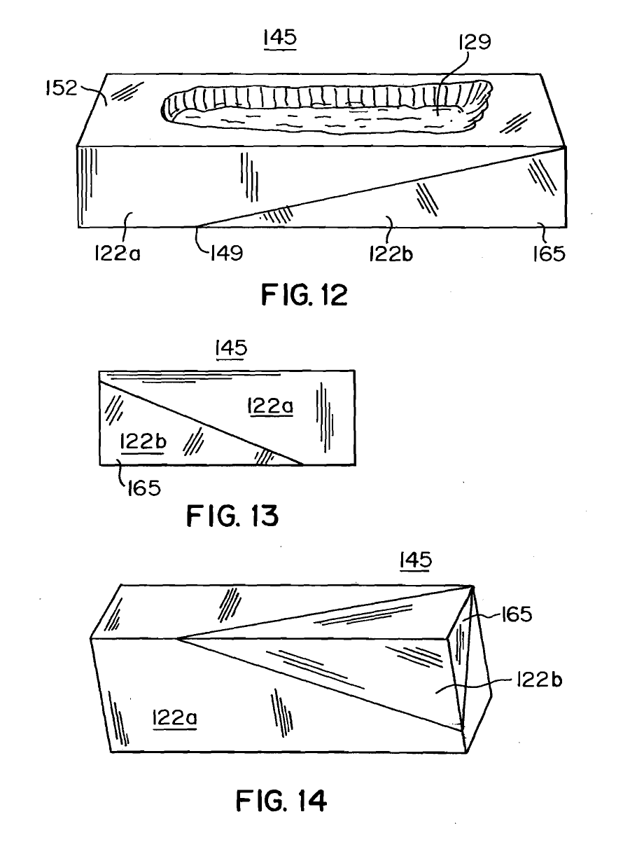

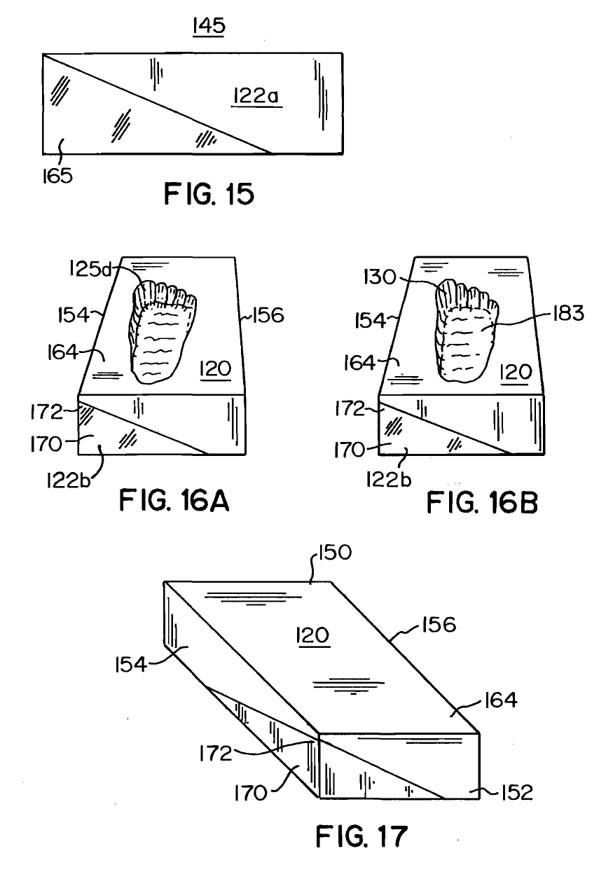

Directing attention to FIGS. 8-15, the ideal impression within a form 120 would be defined by a cavity or impression 123 having depressions 125d, 126d, 127d, 128d, 129d (FIG. 8) representing the depressions caused by the metatarsals 125, 126, 127, 128, 129 (FIG. 6), wherein each of these metatarsal depressions 125d, 126d, 127d, 128d, 129d is in the correct elevation relative to one another with the medial surface of the heel higher than the lateral surface of the heel.

The inventors have realized that a form 120 having dual density foam 122 may be utilized to bias the foot within the form 120 so that the final foot position will produce an impression 123 efficiently suited for fabrication of a matching orthotic with minimum alteration of the impression by the fabricator.

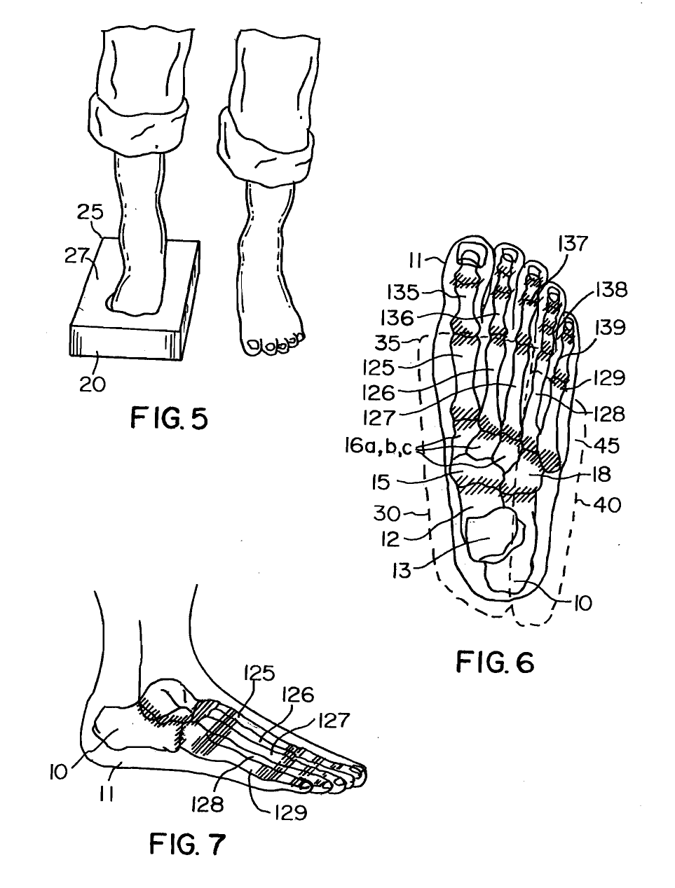

Such an orthosis casting form 120 includes a generally rectangular block 145 of crushable foam 122, wherein the block 145 has a top 147 and a bottom 149 with a front side 150, a rear side 152 and two flanking sides 154, 156 between the top 147 and the bottom 149. The top 147 has a central region 160 adapted to receive the foot 11 (FIGS. 4 and 5) such that under the weight of the user, the foam 122 is crushed and provides an impression 123 of the bottom of the user's foot 11. Of particular interest, the block 145 is made up of at least two different densities of crushable foam 122a, 122b.

It should be understood that the casting form 120 is shown already used with the impression 123 existing. However, one embodiment of the actual invention is directed to the unused form similar in shape to form 20 in FIG. 4.

The central region 160 has a front area 162 proximate to the front side 150 to receive the metatarsals 125, 126, 127, 128, 129 (FIG. 6) and the phalanges 135, 136, 137, 138, 139 (FIG. 6). The central region 160 also has a rear area 164 proximate to the rear side 152 to receive the calcaneus 10. Two densities of foam 122a, 122b are positioned in the front area 162 of the block 145. Foam 122a of lower density is positioned above foam 122b of higher density in the region of the forward portion 45 of the lateral column 40 (FIG. 6) of a foot 11, such that resistance to penetration into the block 145 by the lateral column 40 is greater than resistance to penetration afforded to the medial column 30 (FIG. 6). As illustrated in FIGS. 8-15, the higher density foam 122b has the shape of a wedge 165, wherein, as illustrated in FIG. 9, the highest part of the wedge 165 is in the region of the lateral column 40 of the foot 11 which would be located proximate to the depressions 128d and 129d. As seen in FIG. 9, the wedge 165 tapers downwardly in the direction from the flanking side 156, adjacent to the location of the lateral column 40 (FIG. 6), to the flanking side 154. Additionally, as seen in FIG. 11, the wedge 165 tapers downwardly from the front side 150 to the rear side 152 to insure that the forward portion 45 of the lateral column 40 of the user's foot is urged upwardly.

In general, utilizing this form 120, with an upward force applied to the forward portion 45 of the lateral column 40, the foot becomes “locked” and it is more likely that the final impression 123 will have the first metatarsal depression 125d (FIG. 8) through the fifth metatarsal depression 129d approximately level within the impression 123 as indicated by line 132. While FIGS. 8-14 illustrate a wedge 165, an arrangement to produce this upward force or resistance to the forward portion 45 of the lateral column 40 may be implemented in different ways.

The wedge 165 described herein provides resistance essentially to an entire quadrant of the foot, including the forward portion 45 of the lateral column 40. To the extent that the forward portion 45 of the lateral column 40 may be accurately located on the form 120, then it may not be necessary to utilize a wedge 165 but, in the alternative, a different geometry may be utilized to provide a localized downward resistance to the forward portion 45 of the lateral column 40.

As illustrated in FIG. 19, it should be appreciated that in lieu of a wedge 165 providing resistance to the forward portion 45 of the lateral column 40, it may be entirely possible to utilize a plug 180 extending upwardly from the bottom 249 of the block 245 in the region beneath the forward portion 45 of the lateral column 40 as identified by the foot outline 182. Such a plug, while illustrated as cylindrical, may be another geometric shape capable of selectively adding stiffness below the forward portion 45 of the lateral column 40 when a foot is pressed against the form 220.

The higher density foam 122b may have other shapes including an arch (not shown), wherein the highest part of the shape is in the region of the forward portion 45 of the lateral column 40.

What has so far been discussed is an orthosis casting form 120 comprised of a block 145, wherein the front area 162 has been modified to resist downward motion of the forward portion 45 of the lateral column 40.

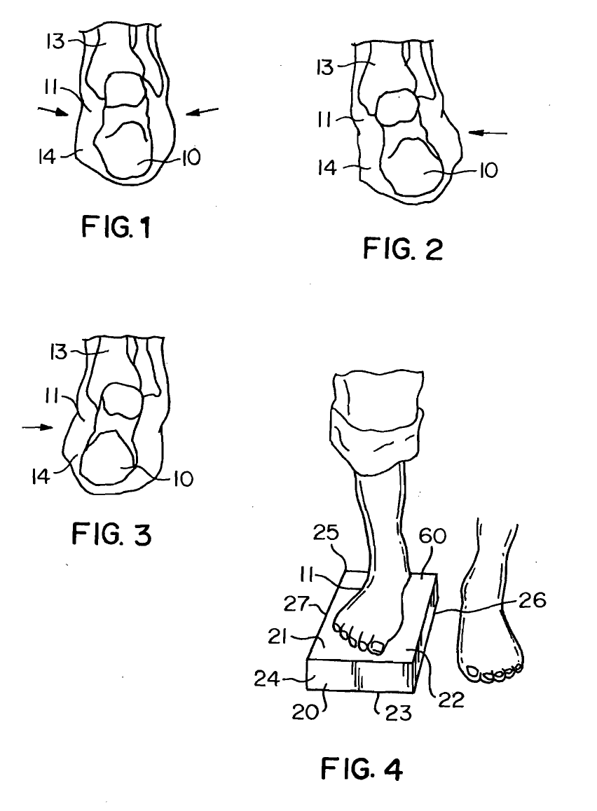

Briefly returning to FIGS. 1-3, as illustrated in FIG. 1, the ideal position of the calcaneus 10 is in alignment with the leg bone 13, or talus bone 12. A pronated foot, as illustrated in FIG. 2, has a calcaneus 10 which is not aligned with the leg bone 13 and causes collapsing of the mid-foot/arch. As a result, the calcaneus 10 is somewhat angled relative to the talus 12. Just as a dual density foam configuration was located beneath the forward portion 45 of the lateral column 40 to position all of the metatarsals 125, 126, 127, 128 129 along a common level plane, a similar concept may be utilized to urge the calcaneus 10 from an everted position, as illustrated in FIG. 2, into the preferred neutral position illustrated in FIG. 1. Directing attention to FIG. 16, the casting form 120 may further include a higher density crushable foam 122b in the rear area 164 to bias the calcaneus 10 in a neutral position as illustrated in FIG. 1.

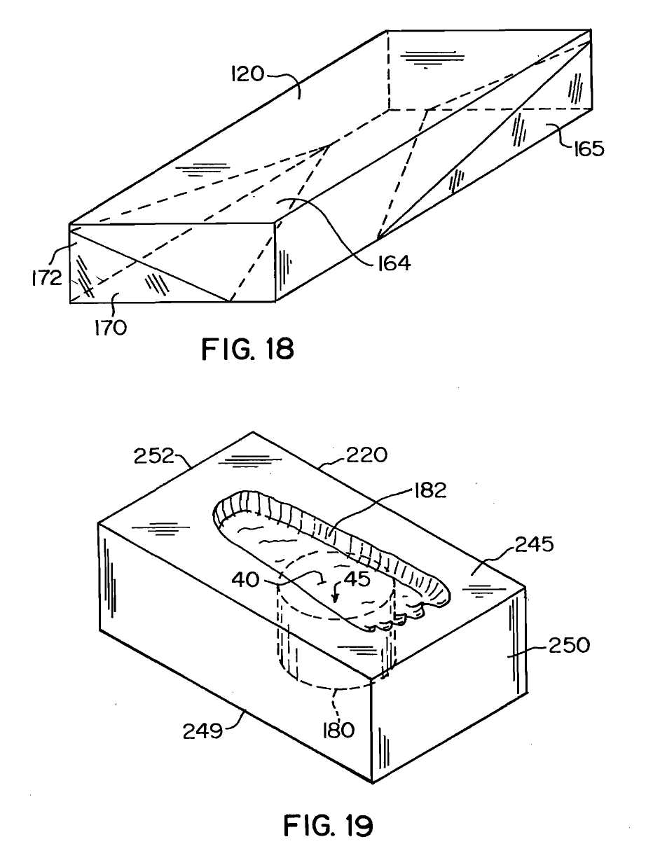

In particular, as illustrated in FIGS. 16-18, higher density foam 122b may have the shape of a wedge 170, wherein the highest part 172 of the wedge 170 is on the side 154 of the block 145 corresponding to the rear area 164 on the flanking side 154 (medial side) of the form 120 to urge the calcaneus 10 to the neutral position. FIG. 17 is a first perspective view of the form illustrated in FIG. 16A and 16B, while FIG. 18 is a second perspective view of the form illustrated in FIG. 16A. These illustrations show the wedge 170 tapers downwardly, not only in the direction from the flanking side 154 (medial side) to the flanking side 156 (lateral side), but also the wedge 170 tapers downwardly in the direction from the rear side 152 (FIG. 17) to the front side 150. As illustrated in FIG. 18, the wedge 170 affecting the calcaneus 10 may work in conjunction with the wedge 165 affecting the forward portion 45 of the lateral column 40.

Whether the cavity 130 is formed with a form 120 having a front wedge 165 or both a front wedge 165 and a rear wedge 170, as illustrated in FIG. 16B, the resultant impression 130 may be filled with a setting foam 183 or a similar space occupying material thereby creating a positive copy of the user's foot in the corrected position. Filling the impression with a setting foam 183 or a similar space occupying material has multiple benefits not only to the customized form described herein, but to standard crushable foam blocks as well. In particular, the individual that takes the foot impression of a patient may not be the same individual that fabricates the orthosis for that patient. More often than not, the “used” foam block with the foot impression of the patient is sent to an individual or company that specializes in fabricating orthosis from the “used” foam blocks. However, the crushable foam is fairly brittle and after a foot impression is made within the block, there may be areas of crushed foam having a thickness of less than 1 inch. Therefore, not only is the crushable foam brittle, but parts of the block are also structurally weak. As a result, when the block, which his usually protected only by a cardboard box, is shipped, the sometimes rigorous shipping experience may cause damage to the block such that the fabricator receives a broken or deformed block. After receiving the “used” block, the fabricator fills the impression with a setting foam to produce a positive upon which the orthosis is formed. If the block is damaged or deformed during handling or shipping, the fabricator must, if possible, reconstruct the block to produce such a positive.

The Applicants have realized that it is very beneficial to fill the impression with settling foam soon after the impression is made so that not only is the block structurally reinforced for shipping and handling, but just as important, the setting foam captures the shape of the impression before the block may be damaged. This provides a very accurate positive that may be used directly by a fabricator to produce an orthosis, thereby eliminating a step for the fabricator while at the same time providing a positive from the impression as it existed just after it was formed. This technique of filling the impression with a setting foam or a similar space occupying material, as mentioned, may be applied to customized forms as described herein or to standard forms.

Additionally, a plug (not shown) similar to plug 180 (FIG. 19) may be used in lieu of the rear wedge 170 in the same fashion the plug 180 is used in lieu of the forward wedge 165.

The crushable foam 122a, 122b discussed herein may be a rigid phenolic foam made of a closed-cell structure and having a density between 0.60 and 0.75 pounds per cubic foot (pcf).

As an example, the foam 122a of the form 120 may have a density of 0.6 pcf while the foam 122b may have a higher density of approximately 0.75 pcf.

It should be appreciated that what has been so far discussed is a foam 122b of higher density, it is also possible to include in place of the foam of high density 122b, a non-resilient rigid element that provides selective resistance to the advancement of the foot 11 within the form 120 to bias the lateral column 40 or to bias the calcaneus 10 into their preferred positions. This substitute material for the foam of higher density 122b may have the same shapes as previously described herein for the foam of higher density 122b, but of a smaller proportion relative to the foam 122 since this material will not flex. It is important to note that whatever material may be used in place of the foam of higher density 122b, it must not be a resilient material because such material after being compressed will rebound, the foam of lower density 122a will always be present. As a result, the rebounding material will most likely push out and crack the foam of lower density 122a.

The subject invention is also directed to a method of making a foot impression form 120 beginning with a block 145 of single density crushable foam 22a having a top 147 and a bottom 149 with sides 50, 52, 54, 56, therebetween. The top 147 has a central region 160 adapted to receive the foot 11 of a user, such that under the weight of the user, the foam 122 is crushed and provides an imprint of the bottom of the user's foot 11.

Directing attention to FIG. 11, the method comprises the steps of removing a portion of the foam 122a from the bottom 149 in the region proximate to the area of the forward portion 45 of the lateral column 40. At least part of the removed portion is replaced with a segment of crushable foam 122b having a greater density, thereby providing greater resistance to penetration of the foam 122b by the fifth metatarsal 129 than by the other metatarsals 125, 126, 127, 128. The method may further include the steps of removing a portion of the foam 122a from the bottom 149 in the region proximate to the area of the calcaneus 10 and replacing at least a part of the removed portion with a segment 122b of crushable foam having a greater density to urge the calcaneus 10 to assume a pronation-neutral position.

In the event the foam of higher density 122b is used to bias the calcaneus 10, the foam of higher density 122b may have the shape of a wedge 170, wherein the highest part 172 of the wedge 170 is on the side 154 of the form 120 corresponding to the first metatarsal 125 to compensate for over pronation.

After the impression 123 (FIG. 9) is formed, the cavity formed may be filled with setting foam or a similar space-occupying material to reinforce the form for shipping and ease of fabrication.

While the apparatus discussed herein involves the use of a foam of lower density 122a and a foam of higher density 122b, it should be fully appreciated that the subject invention is not limited to the selection of foams having only two densities and that two or more foam types may be used alone or in combination to produce the desired results. In one particular example, the higher density foam utilized to bias the forward portion of the lateral column need not have the same density as the foam of higher density utilized to bias the calcaneus into the pronation-neutral position.

While specific embodiments of the invention have been described in detail, it will be appreciated by those skilled in the art that various modifications and alternatives to those details could be developed in light of the overall teachings of the disclosure. The presently preferred embodiments described herein are meant to be illustrative only and not limiting as to the scope of the invention which is to be given the full breadth of the appended claims and any and all equivalents thereof.

1. A logic circuit comprising:
a single-electron spin transistor that includes:
a source, a drain, an island that is provided between the source and the drain, and a gate that is capacitively coupled to the island, a tunnel junction being provided between the source and the island, another tunnel junction being provided between the drain and the island,
at least one of the source, the drain, and the island including a ferromagnetic material having a variable magnetization direction,
wherein a logic circuit function is reconfigured in a nonvolatile manner by changing the variable magnetization direction of the ferromagnetic material of the single-electron spin transistor.

2. The logic circuit as claimed in claim 1, wherein:
the source and the drain of the single-electron spin transistor include ferromagnetic materials magnetized in the same direction; and
the island includes the ferromagnetic material having the variable magnetization direction.

3. The logic circuit as claimed in claim 1, wherein:
the single-electron spin transistor further includes a substrate;
the island, the source, the drain, and the gate are formed on the substrate;
the source, the drain, and the gate are formed on sides of the island; and
the gate is capacitively coupled to the island via a space existing between the gate and the island.

4. The logic circuit as claimed in claim 1, wherein:
the single-electron spin transistor further includes a substrate;
the source, the drain, and the island are stacked on the substrate;
the gate is formed on a side of the island; and
the gate is capacitively coupled to the island via a space existing between the gate and the island.

5. (canceled)

6. The logic circuit as claimed in claim 4, wherein the logic circuit function is a logic threshold value of an inverter circuit.

7. The logic circuit as claimed in claim 4, wherein the logic circuit function is a function of a Boolean logic circuit.

8. The logic circuit as claimed in claim 1, comprising:
a plurality of input terminals; and
a plurality of the single-electron spin transistors,
weighting of analog inputs from the plurality of input terminals to the plurality of single-electron spin transistors is performed with a plurality of gate capacitances of the respective single-electron spin transistors connected to the respective input terminals.

9. The logic circuit as claimed in claim 1, further comprising a first inverter circuit that includes: a first single-electron spin transistor that has a source connected to an output terminal, a gate connected to an input terminal, and a drain connected to a first power supply terminal; and
a second single-electron spin transistor that has a drain connected to the output terminal, a gate connected to the input terminal, and a source connected to a second power supply terminal,
each of the first single-electron spin transistor and the second single-electron spin transistor includes:
an island provided between the source and the drain, the gate is capacitively coupled to the island;
a tunnel junction provided between the source and the island; and
another tunnel junction provided between the drain and the island,
at least one of the source, the drain, and the island including a ferromagnetic material having a variable magnetization direction.

10. The logic circuit as claimed in claim 9, wherein:
when “0” is input to the input terminal, the first single-electron spin transistor is switched on, and the second single-electron spin transistor is switched off; and
when “1” input to the input terminal, the first single-electron spin transistor is switched off, and the second single-electron spin transistor is switched on.

11. The logic circuit as claimed in claim 9, wherein the first inverter circuit switches logic threshold values between a case where a magnetization arrangement of the first single-electron spin transistor is a parallel arrangement while a magnetization arrangement of the second single-electron spin transistor is an antiparallel arrangement, and a case where the magnetization arrangement of the first single-

12. The logic circuit as claimed in claim 9, wherein:
in the first inverter circuit,
the input terminal includes a first input terminal and a second input terminal; and
a combination of an input to the first input terminal and an input to the second input terminal is analog inputs to the first inverter circuit.

13. The logic circuit as claimed in claim 12, wherein:
in the first inverter circuit,
the first input terminal is connected to a first gate of the first single-electron spin transistor and to a first gate of the second single-electron spin transistor; and
the second input terminal is connected to a second gate of the first single-electron spin transistor and to a second gate of the second single-electron spin transistor.

14. The logic circuit as claimed in claim 11, wherein:
in the first inverter circuit,
weighting of an analog input of an input from the first input terminal to the first single-electron spin transistor is substantially the same as weighting of an analog input of the input from the first input terminal to the second single-electron spin transistor; and
weighting of an analog input of an input from the second input terminal to the first single-electron spin transistor is substantially the same as weighting of an analog input of the input from the second input terminal to the second single-electron spin transistor.

15. The logic circuit as claimed in claim 13, wherein:
in the first inverter circuit,
a capacitance value of a first gate capacitance of the first single-electron spin transistor is substantially the same as a capacitance value of a first gate capacitance of the second single-electron spin transistor; and
a capacitance value of a second gate capacitance of the first single-electron spin transistor is substantially the same as a capacitance value of a second gate capacitance of the second single-electron spin transistor.

16. The logic circuit as claimed in claim 14, wherein, in the first inverter circuit, the weighting of the analog inputs of the input from the first input terminal to the first single-electron spin transistor and the second single-electron spin transistor is substantially the same as the weighting of the analog inputs of the input from the second input terminal to the first single-electron spin transistor and the second single-electron spin transistor.

17. The logic circuit as claimed in claim 15, wherein:
in the first inverter circuit,
the capacitance values of the first gate capacitance of the first single-electron spin transistor is substantially the same as the first gate capacitance of the second single-electron spin transistor;
the capacitance values of the second gate capacitance of the first single-electron spin transistor is substantially same as the second gate capacitance of the second single-electron spin transistor.

18. The logic circuit as claimed in claim 16, wherein, in the first inverter circuit, the weighting of the analog inputs of the input from the first input terminal to the first single-electron spin transistor and the second single-electron spin transistor is different from the weighting of the analog inputs from the second input terminal to the first single-electron spin transistor and the second single-electron spin transistor.

19. The logic circuit as claimed in claim 17, wherein, in the first inverter circuit, the capacitance values of the first gate capacitance of the first single-electron spin transistor and the first gate capacitance of the second single-electron spin transistor are different from the capacitance values of the second gate capacitance of the first single-electron spin transistor and the second gate capacitance of the second single-electron spin transistor.

20. The logic circuit as claimed in claim 12, wherein:
the first inverter circuit has a function of a two-input NOR circuit in a case where the magnetization arrangement of the first single-electron spin transistor is the antiparallel arrangement while the magnetization arrangement of the second single-electron spin transistor is the parallel arrangement; and
the first inverter circuit has a function of a two-input NAND circuit in a case where the magnetization arrangement of the first single-electron spin transistor is the parallel arrangement while the magnetization arrangement of the second single-electron spin transistor is the antiparallel arrangement.

21. The logic circuit as claimed in claim 12, wherein the output terminal of the first inverter circuit is connected to an input terminal of a second inverter circuit, to form a two-input OR circuit function and a two-input AND circuit function.

22. The logic circuit as claimed in claim 21, wherein the second inverter circuit is an inverter circuit including a single-electron transistor.

23. The logic circuit as claimed in claim 12, comprising:
the first inverter circuit;
a third single-electron spin transistor that has a source connected to the output terminal of the first inverter circuit and a drain connected to a third power supply terminal; and
a fourth single-electron spin transistor that has a drain connected to the output terminal of the first inverter circuit and a source connected to a fourth power supply terminal,
wherein each of the third single-electron spin transistor and the fourth single-electron spin transistor is the single-electron spin transistor.

24. The logic circuit as claimed in claim 23, wherein:
when “0” is output from the first inverter circuit, the third single-electron spin transistor is switched on, and the fourth single-electron spin transistor is switched off; and
when “1” is output from the first inverter circuit, the third single-electron spin transistor is switched off, and the fourth single-electron spin transistor is switched on.

25. The logic circuit as claimed in claim 23, further comprising:
a third inverter circuit that has an input terminal connected to the first input terminal and the second input terminal of the first inverter circuit, an output terminal connected to a gate of the third single-electron spin transistor, and has a logic threshold value greater than 0.5; and
a fourth inverter circuit that has an input terminal connected to the first input terminal and the second input terminal of the first inverter circuit, an output terminal connected to a gate of the fourth single-electron spin transistor, and has a logic threshold value smaller than 0.5.

26. The logic circuit as claimed in claim 25, wherein weighting of analog inputs from the first input terminal and the second input terminal to the third inverter circuit, and weighting of analog inputs from the first input terminal and the second input terminal to the fourth inverter circuit are substantially the same as weighting of analog inputs from the first input terminal and the second input terminal to the first inverter circuit.

27. The logic circuit as claimed in claim 25, wherein each of the third inverter circuit and the fourth inverter circuit is an inverter circuit including a single-electron transistor.

28. The logic circuit as claimed claim 23, further comprising a fifth inverter circuit that has an input terminal connected to the output terminal of the first inverter circuit.

29. The logic circuit as claimed in claim 28, wherein the fifth inverter circuit is an inverter circuit including a single-electron transistor.

30. The logic circuit as claimed in claim 23, which has a circuit that can realize all Boolean symmetrical functions by switching the magnetization arrangement of each of the first single-electron spin transistor, the second single-electron spin transistor, the third single-electron spin transistor, and the fourth single-electron spin transistor, between the parallel arrangement and the antiparallel arrangement.

31. A single-electron spin transistor comprising:
a substrate;
a source that is formed on the substrate;
an island that is formed on the source and has a tunnel junction between the source and the island;
a drain that is formed on the island and has a tunnel junction between the island and the drain; and
a gate that is formed on a side of the island, and is capacitively coupled to the island via a space existing between the island and the gate,
wherein at least one of the source, the drain, and the island includes a ferromagnetic material having a variable magnetization direction.

32. A single-electron spin transistor comprising:
a substrate;
a drain that is formed on the substrate;
an island that is formed on the drain and has a tunnel junction between the drain and the island;
a source that is formed on the island and has a tunnel junction between the island and the source; and
a gate that is formed on a side of the island, and is capacitively coupled to the island via a space existing between the island and the gate,
wherein at least one of the source, the drain, and the island includes a ferromagnetic material having a variable magnetization direction.

33. The single-electron spin transistor as claimed in claim 31, wherein:
the source and the drain include ferromagnetic materials magnetized in the same direction; and
the island includes the ferromagnetic material having the variable magnetization direction.

34. The single-electron spin transistor as claimed in claim 33, wherein the magnetization direction of the island is changed by injecting carriers from the source or the drain into the island.

35. The single-electron spin transistor as claimed in claim 34, wherein one of the source and the drain has a greater film thickness than the other one of the source and the drain.

36. The single-electron spin transistor as claimed in claim 34, wherein one of the source and the drain has a higher spin polarization than the other one of the source and the drain.

37. The single-electron spin transistor as claimed in claim 33, wherein the island is a carrier-induced ferromagnetic semiconductor film.

38. The single-electron spin transistor as claimed in claim 35, wherein:
the gate is provided on either side of the island; and
the magnetization direction of the island is changed by applying a voltage between the gates and injecting carriers from the source or the drain into the island.

39. The single-electron spin transistor as claimed in claim 38, wherein the voltage applied between the gates is such a voltage as to reduce carrier density in the island.

40. The single-electron spin transistor as claimed in claim 32, wherein:
the source and the drain include' ferromagnetic materials magnetized in the same direction; and
the island includes the ferromagnetic material having the variable magnetization direction.

41. The single-electron spin transistor as claimed in claim 40, wherein the magnetization direction of the island is changed by injecting carriers from the source or the drain into the island.

42. The single-electron spin transistor as claimed in claim 41, wherein one of the source and the drain has a greater film thickness than the other one of the source and the drain.

43. The single-electron spin transistor as claimed in claim 41, wherein one of the source and, the drain has a higher spin polarization than the other one of the source and the drain.

44. The single-electron spin transistor as claimed in claim 40, wherein the island is a carrier-induced ferromagnetic semiconductor film.

45. The single-electron spin transistor as claimed in claim 42, wherein:
the gate is provided on either side of the, island; and
the magnetization direction of the island is changed by applying a voltage between the gates and injecting carriers from the source or the drain into the island.

46. The single-electron spin transistor as claimed in claim 45, wherein the voltage applied between the sates is such a voltage as to reduce carrier density in the island.
